(19) United States
(12) Patent Application Publication
(10) Pub. No.: US 2004/0131547 A1
(43) Pub. Date: Jul. 8, 2004

(54) Contrast agents

(76) Inventors: Balin Balinov, Oslo (NO); Roald Skurtveit, Nittedal (NO); Unni Nordby Wiggen, Rasta (NO); Jonny Ostensen, Oslo (NO)

Correspondence Address: Amersham Health, Inc., IP Department, 101 Carnegie Center, Princeton, NJ, 08540, US

(21) Appl. No.: 10/719,697

(22) Filed: Nov. 21, 2003

Related U.S. Application Data
(63) Continuation of application No. 09/673,160, filed on Dec. 11, 2000, now abandoned.
Application No. 09/673,160 is a 371 of international application No. PCT/GB99/01234, filed on Apr. 22, 1999.
(60) Provisional application No. 60/084,882, filed on May 8, 1998.

(30) Foreign Application Priority Data
Apr. 22, 1998 (GB) 9808581.4

(51) Int. Cl. A61K 49/00

(52) U.S. Cl. 424/9.51; 424/9.52

(57) ABSTRACT

Ultrasound contrast agents of the phase shift colloid type, comprising emulsions of volatile oils in water, are provided with gas-containing nucleation sites associated with (e.g. within) droplets of the dispersed oil phase, in order to enhance efficacy and control of the liquid-to-gas phase transition.

This invention relates to ultrasound imaging, more particularly to novel contrast agent preparations and their use in ultrasound imaging, for example in visualising tissue perfusion.

It is well known that contrast agents comprising dispersions of microbubbles of gases are particularly efficient backscatterers of ultrasound by virtue of the low density and ease of compressibility of the microbubbles. Such microbubble dispersions, if appropriately stabilised, may permit highly effective ultrasound visualisation of, for example, the vascular system and tissue microvasculature, often at advantageously low doses.

The use of ultrasonography to assess blood perfusion (i.e. blood flow per unit of tissue mass) is of potential value in, for example, tumour detection, tumour tissue typically having different vascularity from healthy tissue, and studies of the myocardium, e.g. to detect myocardial infarctions. A problem with the application of existing ultrasound contrast agents to cardiac perfusion studies is that the information content of images obtained is degraded by attenuation caused by contrast agent present in the ventricles of the heart.

In our copending International Patent Publication No. WO-A-9817324, the contents of which are incorporated herein by reference, we have disclosed that ultrasonic visualisation of a subject, in particular of perfusion in the myocardium and other tissues, may be achieved and/or enhanced by means of gas-containing contrast agent preparations which promote controllable and temporary growth of the gas phase in vivo following administration. Such contrast agent preparations may be used to promote controllable and temporary retention of the gas phase, for example in the form of microbubbles, in tissue microvasculature, thereby enhancing the concentration of gas in such tissue and accordingly enhancing its echogenicity, e.g. relative to the blood pool.

Such use of gas as a deposited perfusion tracer differs markedly from existing proposals regarding intravenously administrable microbubble ultrasound contrast agents. Thus it is generally thought necessary to avoid microbubble growth since, if uncontrolled, this may lead to potentially hazardous tissue embolisation. Accordingly it may be necessary to limit the dose administered and/or to use gas mixtures with compositions selected so as to minimise bubble growth in vivo by inhibiting inward diffusion of blood gases into the microbubbles (see e.g. WO-A-9503835 and WO-A-9516467).

In accordance with WO-A-9817324, on the other hand, a composition comprising a dispersed gas phase is coadministered with a composition comprising at least one substance which has or is capable of generating a gas or vapour pressure in vivo sufficient to promote controllable growth of the said dispersed gas phase through inward diffusion thereto of molecules of gas or vapour derived from said substance, which for brevity is hereinafter referred to as a “diffusible component”, although it will be appreciated that transport mechanisms other than diffusion may additionally or alternatively be involved in operation of the invention, as discussed in greater detail hereinafter.

This coadministration of a dispersed gas phase-containing composition and a composition comprising a diffusible component having an appropriate degree of volatility may be contrasted with previous proposals regarding administration of volatile substances alone, e.g. in the form of phase shift colloids as described in WO-A-9416739. Thus the contrast agent preparations of WO-A-9817324 permit control of factors such as the probability and/or rate of growth of the dispersed gas by selection of appropriate constituents of the coadministered compositions, whereas administration of the aforementioned phase shift colloids alone may lead to generation of microbubbles which grow uncontrollably and unevenly, possibly to the extent where at least a proportion of the microbubbles may cause potentially dangerous embolisation of, for example, the myocardial vasculature and brain (see e.g. Schwarz, Advances in Echo-Contrast [1994(3)], pp. 48-49).

It has been found that administration of phase shift colloids alone may not lead to reliable or consistent in vivo volatilisation of the dispersed phase to generate gas or vapour microbubbles. Grayburn et al. in J. Am. Coll. Cardiol. 26(5) [19951], pp. 1340-1347 suggest that preactivation of perfluoropentane emulsions may be required to achieve myocardial opacification in dogs at effective imaging doses low enough to avoid haemodynamic side effects. An activation technique for such colloidal dispersions, involving application of hypobaric forces thereto, is described in WO-A-9640282; typically this involves partially filling a syringe with the emulsion and subsequently forcibly withdrawing and then releasing the plunger of the syringe to generate a transient pressure change which causes formation of gas microbubbles within the emulsion. This is an inherently somewhat cumbersome technique which may fail to give consistent levels of activation.

Again with regard to phase shift colloids, it is stated in U.S. Pat. No. 5,536,489 that emulsions of water-insoluble gas-forming chemicals such as perfluoropentane may be used as contrast agents for site-specific imaging, the emulsions only generating a significant number of image-enhancing gas microbubbles upon application of ultrasonic energy to a specific location in the body which it is desired to image. Our own research has shown, however, that emulsions of volatile compounds such as 2-methylbutane or perfluoropentane give no detectable echo enhancement either in vitro or in vivo when ultrasonicated at energy levels which are sufficient to give pronounced contrast effects using two component contrast agents in accordance with WO-A-9817324.

WO-A-9725097 discloses the administration of aqueous dispersions of superheated droplets of water-immiscible liquids which may be vaporised in vivo under the influence of radiation or ultrasound, which are said to induce homogeneous nucleation of the droplets. The dispersions may be used, inter alia, to form diagnostic is contrast agents or selectively to deliver drugs to a localised body region.

The present invention is based on the finding that volatile emulsions of the phase shift colloid type in which gas-containing heterogeneous nucleation sites are associated with the emulsion droplets possess a number of valuable advantages. In particular, they permit perfusion imaging to be carried out in similar manner to that described in WO-A-9817324, but without the need to administer two separate compositions, thereby facilitating handling of the products. Moreover, factors such as the ultimate size of the gas microbubbles generated by the volatile dispersed phase may be controlled through parameters such as the droplet size of the emulsion and the nature and location of the nucleation sites which may readily be set during manufacture of the contrast agent. Thus the high yield of liquid-to-gas phase transition resulting from the presence of nucleation sites make it possible accurately to forecast the size of the formed microbubbles, so permitting controlled retention with a high safety profile.

Thus according to one aspect of the present invention there is provided an ultrasound contrast agent comprising an injectable oil-in-water emulsion wherein there are gas-containing nucleation sites associated with droplets of the dispersed oil phase.

The invention further provides a method of generating enhanced images of a human or non-human animal subject which comprises the steps of injecting a contrast agent as defined above into the vascular system of said subject and generating an ultrasound image of at least a part of said subject.

The dispersed oil phase may comprise one or more appropriately volatile components where at least one component is at least partially insoluble in and immiscible with water. This component or mixture of components is advantageously a liquid at processing and storage temperature, which may for example be as low as −10° C. if the aqueous phase contains appropriate antifreeze material, while being a gas or exhibiting sufficient vapour pressure, e.g. at least 50 mm Hg, preferably at least 100 mm Hg, at body temperature. Other less volatile substantially water-insoluble and water-immiscible components may if desired also be present in the oil phase.

Appropriate volatile components may, for example, be selected from the various lists of emulsifiable low boiling liquids given in the aforementioned WO-A-9416739, the contents of which are incorporated herein by reference. Specific examples of emulsifiable oil phase components include aliphatic ethers such as diethyl ether; polycyclic oils or alcohols such as menthol, camphor or eucalyptol; heterocyclic compounds such as furan or dioxane; aliphatic hydrocarbons, which may be saturated or unsaturated and straight chained or branched, e.g. as in n-butane, n-pentane, 2-methylpropane, 2-methylbutane, 2,2-dimethylpropane, 2,2-dimethylbutane, 2,3-dimethylbutane, 1-butene, 2-butene, 2-methylpropene, 1,2-butadiene, 1,3-butadiene, 2-methyl-1-butene, 2-methyl-2-butene, isoprene, 1-pentene, 1,3-pentadiene, 1,4-pentadiene, butenyne, 1-butyne, 2-butyne or 1,3-butadiyne; cycloaliphatic hydrocarbons such as cyclobutane, cyclobutene, methylcyclopropane or cyclopentane; and halogenated low molecular weight hydrocarbons (e.g. containing up to 7 carbon atoms). Representative halogenated hydrocarbons include dichloromethane, methyl bromide, 1,2-dichloroethylene, 1,1-dichloroethane, 1-bromoethylene, 1-chloroethylene, ethyl bromide, ethyl chloride, 1-chloropropene, 3-chloropropene, 1-chloropropane, 2-chloropropane and t-butyl chloride. Advantageously at least some of the halogen atoms are fluorine atoms, for example as in dichlorofluoromethane, trichlorofluoromethane, 1,2-dichloro-1,2-difluoroethane, 1,2-dichloro-1,1,2,2-tetrafluoroethane, 1,1,2-trichloro-1,2,2-trifluoroethane, 2-bromo-2-chloro-1,1,1-trifluoroethane, 2-chloro-1,1,2-trifluoroethyl difluoromethyl ether, 1-chloro-2,2,2-trifluoroethyl difluoromethyl ether, partially fluorinated alkanes (e.g. pentafluoropropanes such as 1H,1H,3H-pentafluoropropane, hexafluorobutanes, nonafluorobutanes such as 2H-nonafluoro-t-butane, and decafluoropentanes such as 2H,3H-decafluoropentane), partially fluorinated alkenes (e.g. heptafluoropentenes such as 1H,1H,2H-heptafluoropent-1-ene, and nonafluorohexenes such as 1H,1H,2H-nonafluorohex-1-ene), fluorinated ethers (e.g. 2,2,3,3,3-pentafluoropropyl methyl ether or 2,2,3,3,3-pentafluoropropyl difluoromethyl ether) and, more preferably, perfluorocarbons. Examples of perfluorocarbons include perfluoroalkanes such as perfluorobutanes, perfluoropentanes, perfluorohexanes (e.g. perfluoro-2-methylpentane), perfluoroheptanes, perfluorooctanes, perfluorononanes and perfluorodecanes; perfluorocycloalkanes such as perfluorocyclobutane, perfluorodimethyl-cyclobutanes, perfluorocyclopentane and perfluoromethylcyclopentane; perfluoroalkenes such as perfluorobutenes (e.g. perfluorobut-2-ene or perfluorobuta-1,3-diene), perfluoropentenes (e.g. perfluoropent-1-ene) and perfluorohexenes (e.g. perfluoro-2-methylpent-2-ene or perfluoro-4-methylpent-2-ene); perfluorocycloalkenes such as perfluorocyclopentene or perfluorocyclopentadiene; and perfluorinated alcohols such as perfluoro-t-butanol.

Such at least partially water-insoluble/immiscible volatile substances may contain dissolved materials which significantly increase the vapour pressure of the mixture. Such solute materials include gases such as air; nitrogen; oxygen; carbon dioxide; hydrogen; inert gases such as helium, argon, xenon or krypton; sulphur fluorides such as sulphur hexafluoride, disulphur decafluoride or trifluoromethylsulphur pentafluoride; selenium hexafluoride; optionally halogenated silanes such as methylsilane or dimethylsilane; low molecular weight hydrocarbons (e.g. containing up to 7 carbon atoms), for example alkanes such as methane, ethane, a propane, a butane or a pentane, cycloalkanes such as cyclopropane, cyclobutane or cyclopentane, alkenes such as ethylene, propene, propadiene or a butene, or alkynes such as acetylene or propyne; ethers such as dimethyl ether; ketones; esters; halogenated low molecular weight hydrocarbons (e.g. containing up to 7 carbon atoms); or mixtures of any of the foregoing. Gases such as air, oxygen and carbon dioxide, which have substantial solubility in fluorocarbon liquids, are preferred.

The emulsion will typically be stabilized by one or more surfactants or other encapsulating material. It will be appreciated that the nature of such material may significantly affect factors such as the rate of growth of volatilised gas. In general a wide range of surfactants may be useful, for example selected from the extensive lists given in EP-A-0727225, the contents of which are incorporated herein by reference. Representative examples of useful surfactants include fatty acids (e.g. straight chain saturated or unsaturated fatty acids, for example containing 10-20 carbon atoms) and carbohydrate and triglyceride esters thereof, phospholipids (e.g. a lecithin or a fluorine-containing phospholipid), proteins (e.g. albumins such as human serum albumin), block copolymer surfactants (e.g. polyoxyethylene-polyoxypropylene block copolymers such as Pluronics, or extended polymers such as acyloxyacyl polyethylene glycols, for example polyethyleneglycol methyl ether 16-hexadecanoyloxy-hexadecanoate, e.g. wherein the polyethylene glycol moiety has a molecular weight of 2300, 5000 or 10000), fluorine-containing surfactants (e.g. as marketed under the trade names Zonyl and Fluorad, or as described in WO-A-9639197, the contents of which are incorporated herein by reference), and cationic surfactants, for example comprising one or more quaternary ammonium groups and one or more lipid groups such as long chain (e.g. C₁₀₋₃₀) alkyl or alkanoyl groups.

The emulsion droplets may also be stabilised by wall-forming encapsulating material, so that the dispersed phase is in the form of microcapsules containing the volatile liquid, or by incorporation into porous structures such as latex particles. Representative wall-forming materials include polymers such as polylactic acid, polycaprolactone, polycyanoacrylate and polyesters (e.g. as described in WO-A-9317718).

Nucleation sites may be present within the dispersed oil phase droplets or within surfactant or other encapsulating or stabilizing membranes surrounding the droplets; such membranes may themselves act as nucleation sites per se. Alternatively appropriate nucleation sites may be present in contact with the outside of such membranes.

Where the nucleation sites are present within the oil droplets they may, for example, take the form of dispersed gas microbubbles, e.g. in the form of free microbubbles, surfactant- or lipid-stabilised microbubbles, polymer- or protein-encapsulated microbubbles, gas-containing porous solid microparticles such as aerogels or zeolites, gas entrapped in holes crevices or other irregularities of rough-surfaced solid microparticles, gas-containing polymeric microparticles or gas-containing entities such as fullerenes, clathrates or nanotubes. Such contrast agents may readily be prepared by dispersing the nucleation site-containing material in the oil phase and then generating an oil-in-water emulsion in per se known manner, using one or more appropriate dispersing agents.

In order to facilitate dispersion, the interfacial properties of nucleation sites may, for example, be varied by selection of a dispersing agent for the nucleation sites, or by chemical modification of the nucleation site surface, e.g. by silanisation or plasma modification. The presence of surface irregularities, cavities, edges, crevices or other structural defects which assist a gas phase in spreading on the interface may also be advantageous.

If desired, the nucleation sites may be selected to have interfacial properties which allow them to be located at the water-volatile oil interface. This may, for example, be achieved by choosing a dispersing agent for the nucleation sites which allows the surface of a nucleation site to be partly wetted by both the volatile oil and the aqueous phase. If necessary the surface of the nucleation site may be adjusted by chemical modification (e.g. plasma modification), rinsing etc.

In embodiments of the invention where it is desired that microbubble generation should occur spontaneously in vivo, it is generally preferred that the boiling point of the dispersed oil phase of the emulsion should not exceed 42° C., i.e that the sum of partial pressures from the volatile component(s) of the oil phase should exceed one atmosphere at 42° C.

In other embodiments of the invention microbubbles may be generated either in vivo or immediately prior to injection by appropriate temperature and/or pressure modifications or application of external activating influences such as sound, ultrasound or radiation. When external activating influences are applied, emulsions in which the oil phase has a higher boiling point, e.g. up to 60° C., may also be useful, since the external activation may cause sufficient evaporation of the oil phase in vivo despite its boiling point being more removed from body temperature.

Microbubbles generated from contrast agents according to the present invention are characterised by is a readily controllable rate of growth and final size; they may, for example be designed to grow to a size of e.g. 10-20 μm in order to exhibit controlled retention in tissue microvasculature, e.g. in the myocardium, or may be designed to grow to a size of e.g. 1-7 μm so that they behave as free-flowing contrast agents.

It will be appreciated that liquid-to-gas phase shift in emulsion droplets in the presence of nucleation sites ensures a highly efficient and rapid transformation of the liquid, hence limiting diffusion of volatile substance between separated particles and thus limiting uncontrolled bubble growth. In this respect, the material inside one emulsion droplet may be transformed to one bubble. Assuming a gas which can be described by the ideal gas law [Equation (1)],

pV=nRT (1)

where n is number of moles of substance to make one bubble and is related to the radius of the emulsion droplet, rₑ, by Equation (2)

$$n = \frac{d \cdot {V}_{e}}{{M}_{W}} = \frac{d}{{M}_{W}} \cdot \frac{4}{3} π {r}_{e}^{3} \qquad ( 2 )$$

where d is the density of the liquid phase, M_{w} is the molecular weight of the volatile substance and Vₑ is the volume of the liquid droplet, then inserting Equation (2) into the ideal gas law Equation (1), and expressing the volume V of the obtained gas bubble by its radius r_{b} gives;

$${r}_{b} = {r}_{e} \sqrt[3]{\frac{R T d}{p {M}_{W}}} \approx 0.29 \cdot {r}_{e} \sqrt[3]{\frac{d}{{M}_{W}}} \qquad ( 3 )$$

For a typical volatile solvent, for example perfluoropentane, d is 1.66 g/ml, M_{w}=288 g/mol and using T=298 K and p=1 atm, gives r_{b}≈5.2rₑ. The emulsion droplet should therefore have a size slightly below 2 μm in order to give a microbubble of size 10 μm which is therefore capable of temporary retention.

For the nucleation site to occupy 50% of such an emulsion droplet, its size should be below 1.6 μm. More preferably the nucleation site should occupy less than 20% of the emulsion droplet, so that its size should be below 1.2 μm; even more preferably, the nucleation site should occupy less than lot of the liquid volume and so should have a size below 1 μm.

In order to ensure boiling of a sufficiently high number of emulsion droplets, a sufficiently high number of nucleation sites should be added. The nucleation sites will be distributed on the liquid carrier particles by simple Boltzmann distribution, and calculations may be made to estimate the amount of nucleation sites to be added for a given fraction of the liquid carrier particles to contain at least one nucleation site.

Activation of the phase transition from liquid to gas may be obtained by simply heating to temperatures above the boiling point of the volatile liquid. In order for phase transition to be activated on injection by utilizing the increase in temperature to body temperature, a volatile oil with boiling point below body temperature should be used. However, since bubble nucleation rate may be low also at elevated temperatures, the volatile liquid may have a boiling point well below body temperature. In such a superheated dispersion, presence of nucleation sites may lower the barrier for phase shift so that nucleation can be induced by means of an external influence.

Products in which gas formation is activated by ultrasonication or like treatment may be particularly advantageous in that they may be highly storage-stable prior to activation and use.

It will be appreciated that the dispersed gas content of contrast agents according to the invention will tend to be temporarily retained in tissue in concentrations proportional to the regional rate of tissue perfusion. Accordingly, when using ultrasound imaging modalities such as conventional or harmonic B-mode imaging where the display is derived directly from return signal intensities, images of such tissue may be interpreted as perfusion maps in which the displayed signal intensity is a function of local perfusion. This is in contrast to images obtained using free-flowing contrast agents, where the regional concentration of contrast agent and corresponding return signal intensity depend on the actual blood content rather than the rate of perfusion of local tissue.

In cardiac studies, where perfusion maps are derived from return signal intensities in accordance with this embodiment of the invention, it may be advantageous to subject a patient to physical or pharmacological stress in order to enhance the distinction, and thus the difference in image intensities, between normally perfused myocardium and any myocardial regions supplied by stenotic arteries. As is known from radionuclide cardiac imaging, such stress induces vasodilatation and increased blood flow in healthy myocardial tissue, whereas blood flow in underperfused tissue supplied by a stenotic artery is substantially unchanged since the capacity for arteriolar vasodilatation is already exhausted by inherent autoregulation seeking to increase the restricted blood flow.

The application of stress as physical exercise or pharmacologically by administration of adrenergic agonists may cause discomfort such as chest pains in patient groups potentially suffering from heart disease, and it is therefore preferable to enhance the perfusion of healthy tissue by administration of a vasodilating drug, for example selected from adenosine, dipyridamole, nitroglycerine, isosorbide mononitrate, prazosin, doxazosin, dihydralazine, hydralazine, sodium nitroprusside, pentoxyphylline, amelodipine, felodipine, isradipine, nifedipine, nimodipine, verapamil, diltiazem and nitrous oxide. In the case of adenosine this may lead to in excess of fourfold increases in coronary blood flow in healthy myocardial tissue, greatly increasing the uptake and temporary retention of contrast agents in accordance with the invention and thus significantly increasing the difference in return signal intensities between normal and hypoperfused myocardial tissue. Because an essentially physical entrapment process is involved, retention of contrast agents according to the invention is highly efficient; this may be compared to the uptake of radionucleide tracers such as thallium 201 and technetium sestamibi, which is limited by low contact time between tracer and tissue and so may require maintenance of vasodilatation for the whole period of blood pool distribution for the tracer (e.g. 4-6 minutes for thallium scintigraphy) to ensure optimum effect. The contrast agents of the invention, on the other hand, do not suffer such diffusion or transport limitations, and since their retention in myocardial tissue may also rapidly be terminated by the methods described above, the period of vasodilatation needed to achieve cardiac perfusion imaging in accordance with this embodiment of the invention may be very short, for example less than one minute. This will reduce the duration of any possible discomfort caused to patients by administration of vasodilator drugs.

In view of the fact that the required vasodilatation need only be short lasting, adenosine is a particularly useful vasodilating drug, being both an endogenous substance and having a very short-lasting action as evidenced by a blood pool half-life of only a few seconds. Vasodilatation will accordingly be most intense in the heart, since the drug will tend to reach more distal tissues in less than pharmacologically active concentrations. It will be appreciated that because of this short half-life, repeated injection or infusion of adenosine may be necessary during cardiac imaging in accordance with this embodiment of the invention; by way of example, an initial administration of 150 μg/kg of adenosine may be made substantially simultaneously with administration of the contrast agent composition, followed 10 seconds later by slow injection of a further 150 μg/kg of adenosine, e.g. over a period of 20 seconds.

The contrast agents of the invention may usefully be employed in therapeutic applications such as drug delivery agents. Thus hydrophobic drugs may be dissolved in the volatile oil phase to achieve an advantageously high drug load. Therapeutics may also be incorporated into any encapsulating membrane or may be dissolved in the aqueous carrier phase. Therapeutics may also be present as nano- or micro-sized particles which may function as additional nucleation sites.

Without being bound with theoretical considerations, it is believed that evaporation of the volatile oil droplets will accelerate release of a dissolved therapeutic drug due to the increased concentration of drug in the liquid droplet, which may easily exceed the solubility level. Drug uptake may be also enhanced due to local shear and effects from “microstreaming” induced from the microbubble formation.

According to yet another aspect of the current invention, the induced liquid-to-gas transition may be utilised in applications such as ultrasound therapy. Thus, for example, the liquid-to-gas phase transition may provide microbubbles with a size sufficient to embolize capillaries, and hence may block blood flow to a site of interest, for instance a tumour, following appropriate application of localised ultrasound. The microbubbles may also absorb ultrasound energy and hence may provide heating of a site of interest which may be utilised in hyperthermia treatment. Furthermore, the liquid to gas transition may be very rapid, providing shear forces or microstreaming with a damaging effect on surrounding cells; this may be useful in cell killing, for example in treatment of cancer.

The following non-limitative Examples serve to illustrate the invention.

### EXAMPLE 1

A spatula edge of micronised kaolin is added to 2 ml perfluoropentane (b.p. 28° C.) containing 0.2 ml Fluorad™ FC-171 surfactant. A milky white dispersion is obtained after gently shaking by hand. 0.1 ml of the above dispersion is mixed with 1 ml water by shaking on an Espe Capmix® for 30 seconds, yielding an emulsion with droplet size slightly above 1 μm.

A droplet of the emulsion is placed on a cooling/heating stage, and heated to 37° C. while following the process in a microscope. Several 10 μm droplets appear, demonstrating a rapid liquid-to-gas phase shift in the emulsion droplets.

A tube containing the emulsion is dipped in a water bath maintained at 37° C. so that only one part of the emulsion is heated. The turbidity immediately increases significantly in that part of the emulsion which is heated relatively to the non-heated emulsion, demonstrating the formation of small gas bubbles after heating.

### EXAMPLE 2

A spatula edge of micronised zeolite is added to 2 ml perfluoropentane (b.p. 28° C.) containing 0.2 mg perfluorooctanoic acid. The sample is sonicated using a Branson W385 sonicator horn at 50% output power for two minutes while keeping the sample in an ice bath. 0.1 ml of the above dispersion is mixed with 1 ml water by shaking on an Espe Capmix® for 45 seconds, yielding an emulsion.

A sample of the emulsion (1 μl) is suspended in Isoton II (55 ml) at room temperature, and acoustic attenuation is measured as a function of time using two broadband transducers with centre frequencies of 3.5 MHz and 5.0 MHz respectively, in a pulse-echo technique. The acoustic attenuation is weak. The sample is then heated step-wise and acoustic attenuation is measured for each temperature. When the sample temperature is around 30° C., a substantial increase in acoustic attenuation can be observed. This experiment demonstrates how a nucleation site-containing emulsion of a volatile substance can transform to a microbubble dispersion around its boiling point. It also demonstrates the change in acoustic properties and the product's usefulness as an ultrasound contrast agent.

### EXAMPLE 3 (COMPARATIVE)

Example 2 is repeated without adding micronised zeolite to the perfluoropentane phase. When characterising the emulsion using the acoustic attenuation measurement technique, heating to temperatures well above 40° C. leads only to a slight increase in acoustic attenuation. This demonstrates the requirement for nucleation sites to be associated with the dispersed phase.

### EXAMPLE 4

a) 5 ml of a 5% w/v solution of the polymer from Example 2(a) of WO-A-9607434 in (−)-camphene, maintained at 60° C., is added to 15 ml of a 5% w/v solution of human serum albumin in water at the same temperature. The mixture is mixed hot with an Ultra Turax T25 mixer at 20,000 rpm for 1 minute. Thereafter, the emulsion is homogenised at 60° C. using an Emulsiflex C5 high-pressure homogeniser, operating at a peak pressure of 200,000 kPa and allowing five passes of the sample. The median size of the obtained emulsion is around 300 nm. The emulsion is then frozen on a dry ice/methanol bath and lyophilised for 48 hours, giving a white powder. Electron microscopy indicates the formation of gas-filled nanocapsules. The polymer particles are dispersed in water and excess human serum albumin is removed by dialysis. The remaining polymer nanocapsules are dried under reduced pressure.

b) A spatula edge of the washed, hollow polymer-stabilised nanocapsules from (a) above is added to 2 ml perfluorodimethylcyclobutane (b.p. 45° C.) containing 0.2 ml perfluorooctanoic acid. The sample is shaken on a laboratory shaker for one hour, yielding a dispersion of gas-filled nanocapsules dispersed in perfluorodimethylcyclobutane. 0.1 ml of the above dispersion is mixed with 1 ml water by shaking on an Espe Capmix® for 45 seconds, yielding an emulsion.

c) A droplet of the emulsion from (b) above is placed on a cooling/heating stage and heated to 50° C., while following the process in a microscope. Several 10 μm droplets appear when the temperature passes 45° C., demonstrating a rapid liquid-to-gas phase shift in the emulsion droplets.

d) A tube containing diluted emulsion from (b) above is dipped in a water bath maintained at 50° C., so that only part of the emulsion is heated. The turbidity immediately increases significantly in the heated part of the emulsion relative to the non-heated part, demonstrating the formation of small gas bubbles on heating.

e) A sample of the emulsion from (b) above (1 μl) is suspended in Isoton II (55 ml) at room temperature, and acoustic attenuation is measured as a function of time using two broadband transducers with centre frequencies of 3.5 MHz and 5.0 MHz respectively, in a pulse-echo technique. The acoustic attenuation is weak. The sample is then heated step-wise and acoustic attenuation is measured for each temperature. When the sample temperature passes 35-40° C., a substantial increase in acoustic attenuation can be observed. This experiment demonstrates how a nucleation site-containing emulsion of a volatile substance can transform to a microbubble dispersion well below its boiling point when the emulsion is exposed to external ultrasound. It also demonstrates the change in acoustic properties and the product's usefulness as an ultrasound contrast agent.

### EXAMPLE 5

A dog is anaesthetised, a mid-line sternotomy is performed, and the anterior pericardium is removed. Mid-line short-axis B-mode imaging of the heart is performed through a low-attenuating 30 mm silicone rubber spacer, using an ATL HDI-3000 scanner equipped with a P3-2 transducer. The framerate is 40 Hz and the mechanical index is 1.1. An amount of the polymer nanocapsule-containing perfluorodimethylcyclobutane emulsion of Example 4(b), corresponding to 0.2 μl perfluorodimethylcyclobutane/kg body weight, is injected intravenously into the dog. A substantial rise in echo intensity from the myocardium is seen, starting some 20 seconds after the injection and lasting for 20 minutes. The increase in myocardial opacification is seen at a time when the ventricles are almost emptied of contrast. The observed efficacy is therefore due to microbubbles retarded in the myocardium.

### EXAMPLE 6 (COMPARATIVE)

Example 5 is repeated except that a perfluorodimethyl-cyclobutane emulsion phase is used without added polymeric nanocapsules. In vivo ultrasound imaging indicates limited acoustic efficacy of the emulsion. This comparative experiment shows the necessity for gas-filled nucleation site associated with the emulsion droplets.

1. An ultrasound contrast agent comprising an injectable oil-in-water emulsion wherein there are gas-containing nucleation sites associated with droplets of the dispersed oil phase.

2. A contrast agent as claimed in claim 1 wherein the nucleation sites are present within dispersed oil phase droplets.

3. A contrast agent as claimed in claim 2 wherein the nucleation sites comprise free gas microbubbles, surfactant- or lipid-stabilised gas microbubbles, polymer- or protein-encapsulated gas microbubbles, gas-containing porous solid microparticles, gas-containing rough-surfaced solid microparticles, gas-containing polymeric microparticles, or gas-containing fullerenes, clathrates or nanotubes.

4. A contrast agent as claimed in claim 1 wherein nucleation sites are present within membranes stabilising the dispersed oil phase droplets or in contact with the outside of such membranes.

5. A contrast agent as claimed in any of the preceding claims wherein the oil phase comprises one or more components selected from aliphatic ethers, polycyclic oils and alcohols, heterocyclic compounds, aliphatic hydrocarbons, cycloaliphatic hydrocarbons and halogenated hydrocarbons, said component(s) having a boiling point not exceeding 60° C.

6. A contrast agent as claimed in claim 5 wherein the oil phase comprises one or more perfluorocarbons.

7. A contrast agent as claimed in claim 6 wherein said perfluorocarbon is selected from perfluorobutanes, perfluoropentanes, perfluorohexanes, perfluorocyclobutane, perfluorodimethylcyclobutanes, perfluorocyclopentane, perfluoromethylcyclopentane, perfluorobutenes, perfluorobutadienes, perfluoropentenes, perfluorohexenes, perfluorocyclopentene, perfluorocyclopentadiene and perfluoro-t-butanol.

8. A contrast agent as claimed in any of the preceding claims wherein the oil phase contains a gaseous solute.

9. A contrast agent as claimed in claim 8 wherein the oil phase comprises air, oxygen or carbon dioxide dissolved in a liquid fluorocarbon.

10. A contrast agent as claimed in any of the preceding claims wherein the dispersed oil phase droplets are stabilised by a surfactant selected from fatty acids, carbohydrate and triglyceride esters of fatty acids, phospholipids, proteins, block copolymer surfactants, fluorine-containing surfactants and cationic surfactants.

11. A contrast agent as claimed in any of claims 1 to 9 wherein the dispersed oil phase droplets are stabilised by polymeric wall-forming encapsulating material or by incorporation into porous latex particles.

12. A contrast agent as claimed in any of the preceding claims wherein the oil phase has a boiling point not exceeding 42° C.

13. A combined preparation for simultaneous, separate or sequential use as a contrast agent in ultrasound imaging, said preparation comprising:
i) a contrast agent as claimed in any of the preceding claims, and
ii) a vasodilator drug.

14. A combined preparation as claimed in claim 13 wherein said vasodilator drug is adenosine.

15. A drug delivery agent comprising a contrast agent as claimed in any of claims 1 to 12 together with a therapeutic drug.

16. A drug delivery agent as claimed in claim 15 wherein a hydrophobic drug is dissolved in the oil phase.

17. A drug delivery agent as claimed in claim 15 wherein the drug is present as nano- or micro-sized particles.

18. A method of generating enhanced images of a human or non-human animal subject which comprises the steps of injecting a contrast agent as claimed in any of claims 1 to 14 into the vascular system of said subject and generating an ultrasound image of at least a part of said subject.

19. A method as claimed in claim 18 wherein microbubble growth from the contrast agent is activated within the subject by application of external activation.

20. A method as claimed in claim 19 wherein said external activation comprises ultrasound irradiation.

21. Use of a contrast agent as claimed in any of claims 1 to 12 in ultrasound therapy.

22. Use as claimed in claim 21 wherein said therapy involves cell killing or blocking of blood flow to a site of interest.
